(19) United States
(12) Patent Application Publication
(10) Pub. No.: US 2007/0173688 A1
(43) Pub. Date: Jul. 26, 2007

(54) Laproscope with flexible binocular camera

(76) Inventor: Young-Woo Kim, Seoul (KR)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 10/577,170

(22) PCT Filed: Jul. 23, 2004
(86) PCT No.: PCT/KR04/01849

§ 371 Date: Dec. 13, 2006

(30) Foreign Application Priority Data
Jul. 23, 2003 (KR) 10-2003-0050586

(51) Int. Cl. A61B 1/04; A61B 1/06

(52) U.S. Cl. 600/111; 600/166

(57) ABSTRACT

Disclosed herein is a stereoscopic laparoscope apparatus comprising a laparoscope, a computer adapted to convert and store image information of the patient's affected part from the laparoscope, and a monitor used to output the image information. The laparoscope comprises a supporting unit including a manipulator, and a pair of parallel left and right supporting rods, a flexible tube unit including a pair of left and right flexible tubes, which are adapted to be spaced apart from each other within a predetermined angular range, and a binocular camera assembly including a pair of left and right cameras installed at the tip end of the flexible tube unit so that they take images of the affected part in the abdominal cavity under operation of the manipulator. With such a configuration, the image information of the patient's affected part can be processed into stereoscopic photographs, resulting in precise diagnosis and laparoscopic surgery.

### TECHNICAL FIELD

The present invention relates to stereoscopic laparoscopes, and more particularly to stereoscopic laparoscopes in which dual cameras are arranged in parallel at tip ends of respective supporting rods to achieve a stereoscopic vision, the dual cameras being adapted to be spaced apart from each other by a predetermined distance in the abdominal cavity of a patient through manipulation of a lever or button so as to impart the stereoscopic vision similar to that of a human, such that obtained image information of the patient's affected part can be processed into stereoscopic photographs by a computer and displayed on a monitor, resulting in precision in video diagnosis as well as accuracy and convenience in laparoscopic surgery.

### BACKGROUND ART

In general, laparoscopic surgery is surgery conducted within the abdominal cavity of a patient through a natural opening or small incision while viewing the sight in the abdominal cavity. The laparoscopic surgery is widely used in various surgical procedures, such as cholecystectom, appendectomy, gastrectomy, colon resection, etc., and is also applicable in the field of urology and obstetrics.

In such a laparoscopic surgery, a laparoscope is usually used to provide inevitable information for diagnosing and treatment related to internal organs of the human body. Conventionally, the laparoscope comprises a micro camera so that, in an inserted state in the internal organs of the human body, it can allow an operator to observe image information obtained from the micro camera using a monitor.

As well known, the laparoscopic surgery is conducted using an endoscope, medical laser device and other small diagnostic instruments without requiring cutting the abdomen open. In such a system using endoscopes, peripheral devices thereof play an important role by visualizing the accurate state of the affected part to an operator.

For producing a stereoscopic image, conventional laparoscopes utilize a technique wherein two planar images of the same scene, taken as light is irradiated and reflected to an object at slightly different angles, are combined, or a technique wherein two planar images are divided from a single planar image, and then one of the divided planar images is exposed to a stereoscope after a lapse of time.

FIG. 1 is a front view illustrating a conventional stereoscopic laparoscope apparatus. FIGS. 2 and 3 are respectively left-side and right-side views of the conventional laparoscope apparatus. FIG. 4 is a partial sectional view of the conventional laparoscope apparatus.

Now, the schematic structure of the conventional stereoscopic laparoscope apparatus will be explained with reference to FIGS. 1 to 4.

The conventional laparoscope apparatus generally comprises a laparoscope 10, a washing liquid injection port 20, a light injection port 30, a stereoscopic camera coupling portion 40, and a stereoscopic camera connecting portion 50.

Here, the laparoscope 10 contains left optics 11L and 13L and right optics 11R and 13R, optical fibers 14 as injection means of light required to illuminate the interior of the human body, a tube (not shown) for guiding washing liquid to the left and right objective lens 11L and 11R in order to prevent their fogging and pollution, and a washing liquid spray nozzle 12 installed at a distal end of the washing liquid guiding tube.

To the washing liquid injection port 20 is connected a device for injecting the washing liquid at a high pressure so that the washing liquid can be sprayed from the nozzle 12, and to the light injection port 30 is connected a light emitter used to produce light as an optical source. Further, a stereoscopic camera is connected to both the camera coupling portion 40 and the connecting portion 50. In the above description, not designated reference numeral 41 and 51 respectively indicate a coupling groove and a position fixing groove for use in the coupling of the stereoscopic camera, and not designated reference numeral 52L and 52R indicate lens for focusing left and right images of the same scene to left and right image pickup devices included in the stereoscopic camera.

Now, the operation of the above described conventional stereoscopic laparoscope apparatus will be explained.

First, if the laparoscope 10 is introduced into the abdominal cavity of a patient, the light emitted from the optical source is irradiated to the affected part of the patient successively through the light injection port 30 and the optical fibers contained in the laparoscope 10. That is, the light is irradiated from distal ends of the optical fibers 14 (See FIG. 2) to the affected part. The irradiated light is reflected from the affected part, and enters to the left and right objective lens 11L and 11R.

In this way, the light, containing images of the affected part taken at different angles, is transmitted to the left and right image pickup devices of the stereoscopic camera through the left and right optics 13L and 13R and the left and right lens 52L and 52R, thereby allowing resultant left and right images entered in the stereoscopic camera to be displayed on a monitor as stereoscopic optical images. As a result, the operator, namely, the surgeon can perform laparoscopic surgery in the cavity of the patient while observing the stereoscopic optical images of the patient's affected part.

The above described conventional stereoscopic laparoscope apparatus, however, is disadvantageous since it requires to install a plurality of the optics 11L, 11R, 13L and 13R in the narrow laparoscope 10 for the individual guiding of the left and right images. The optics 11L, 11R, 13L and 13R are difficult in manufacture due to their small size. Even if they are laboriously manufactured, it is very difficult to arrange them at accurate positions required to achieve perfect functionality of the optics. Such a difficulty in manufacture and arrangement, consequently, increases manufacturing costs of the optics.

Meanwhile, one of the above described techniques, wherein a single planar image is divided into two planar images, has been conventionally applied to stereoscopic video apparatuses or telescopes, but was not applied to stereoscopic laparoscope apparatuses.

That is, such a technique cannot be directly applied to endoscopes, and has a difficulty in optical control of optics consisting of beam splitters, concave lens, and eyepieces. Further, the fact that left and right optics are arranged in two rows within a single tubular body of the laparoscope complicate the overall structure thereof and excessively increases the size and complicity of such a three-dimensional image producing apparatus.

FIG. 5 is a view illustrating another embodiment of a laparoscope in accordance with the prior art. As shown in FIG. 5, the multi-directional imaging laparoscope comprises a camera 38, which is connected to the tip end of a supporting rod 34 by interposing a flexible tube 36 so that the direction thereof is convertible into four directions.

In such a multi-directional imaging laparoscope, although it is easy to take images since the camera 38 is convertible in its image taking direction into four directions, it exhibits problems of monocular structure and cannot achieve clear stereoscopic image of the affected part via a monitor.

### DESCRIPTION OF THE DRAWINGS

The above and other objects, features and other advantages of the present invention will be more clearly understood from the following detailed description taken in conjunction with the accompanying drawings, in which:

FIG. 1 is a front view illustrating a conventional stereoscopic laparoscope apparatus;

FIG. 2 is a left-side view of the conventional stereoscopic laparoscope apparatus;

FIG. 3 is a right-side view of the conventional stereoscopic laparoscope apparatus;

FIG. 4 is a partial sectional view of the conventional stereoscopic laparoscope apparatus;

FIG. 5 is a perspective view illustrating another embodiment of a conventional laparoscope;

FIG. 6 is a block diagram illustrating a stereoscopic laparoscope apparatus having a spacing adjustable binocular camera assembly in accordance with the present invention;

FIG. 7 is a perspective view schematically illustrating the stereoscopic laparoscope apparatus having a spacing adjustable binocular camera assembly in accordance with the present invention;

FIG. 8 is a partially broken away sectional view illustrating a state wherein dual left and right cameras adjoin each other; and

FIG. 9 is a partially broken away sectional view illustrating a state wherein the dual left and right cameras are spaced apart from each other.

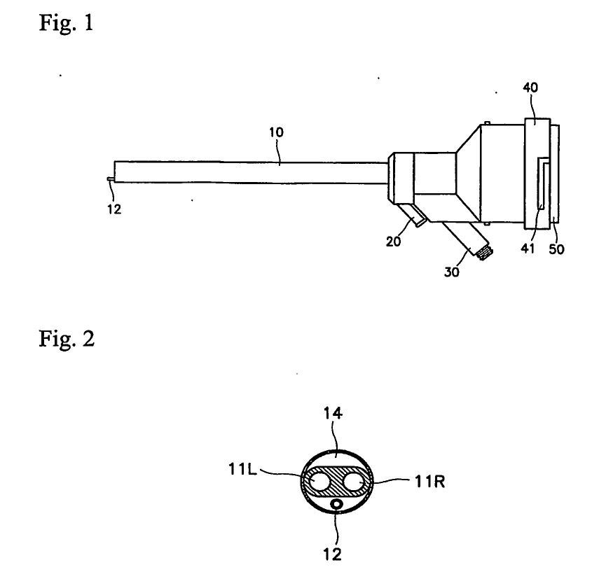

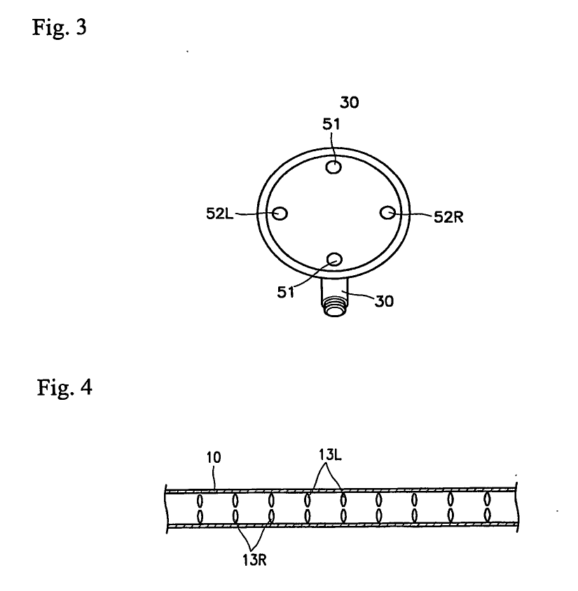

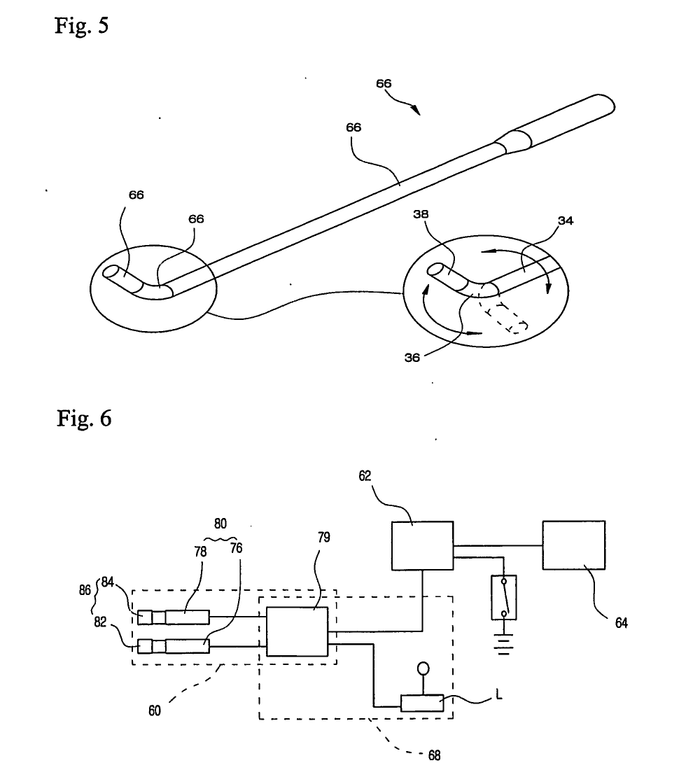

### DISCLOSURE OF INVENTION

### Technical Problem

Therefore, the present invention has been made in view of the above problems, and it is an object of the present invention to provide a stereoscopic laparoscope in which dual cameras are arranged in parallel at tip ends of respective supporting rods to achieve a stereoscopic vision, the dual cameras being adapted to be spaced apart from each other by a predetermined distance in the abdominal cavity of a patient through manipulation of a lever or button so as to impart the stereoscopic vision similar to that of a human, such that obtained image information of the patient's affected part can be processed into stereoscopic photographs by a computer and displayed on a monitor, resulting in precision in video diagnosis as well as accuracy and convenience in laparoscopic surgery.

### Technical Solution

In accordance with an aspect of the present invention, the above and other objects can be accomplished by the provision of a stereoscopic laparoscope apparatus comprising a laparoscope, a computer adapted to convert and store image information of the patient's affected part inputted via the laparoscope, and a monitor used to output the image information converted by the computer, the laparoscope comprising: a supporting unit including a manipulator provided in a body of the laparoscope electrically connected to the computer, and a pair of parallel left and right supporting rods located at one side of the manipulator and having a predetermined length and diameter; a flexible tube unit including a pair of left and right flexible tubes, which are adapted to be spaced apart from each other within a predetermined angular range according to electric signals generated from the manipulator installed at the tip end of the supporting unit; and a binocular camera assembly including a pair of left and right cameras installed at the tip end of the flexible tube unit so that they take images of the affected part in the abdominal cavity under operation of the manipulator.

### Advantageous Effects

As can be seen from the above description, the present invention employs a binocular camera assembly consisting of left and right cameras, and these cameras can be spaced apart from each other inside the abdominal cavity of a patient so as to realize a stereoscopic vision similar to that of a human. This has the effect of facilitating acquisition of a stereoscopic image. In comparison to a monocular camera wherein the process of image information related to the patient's affected part depends on the proficiency of an operator or surgeon, the binocular camera of the present invention, providing a stereoscopic vision, enables a relatively wide area of the affected part to be rapidly and accurately examined even by a beginner, resulting in rapid and precise laparoscopic surgery.

### BEST MODE FOR CARRYING OUT THE INVENTION

Now, a preferred embodiment of the present invention will be explained in more detail with reference to the accompanying drawings.

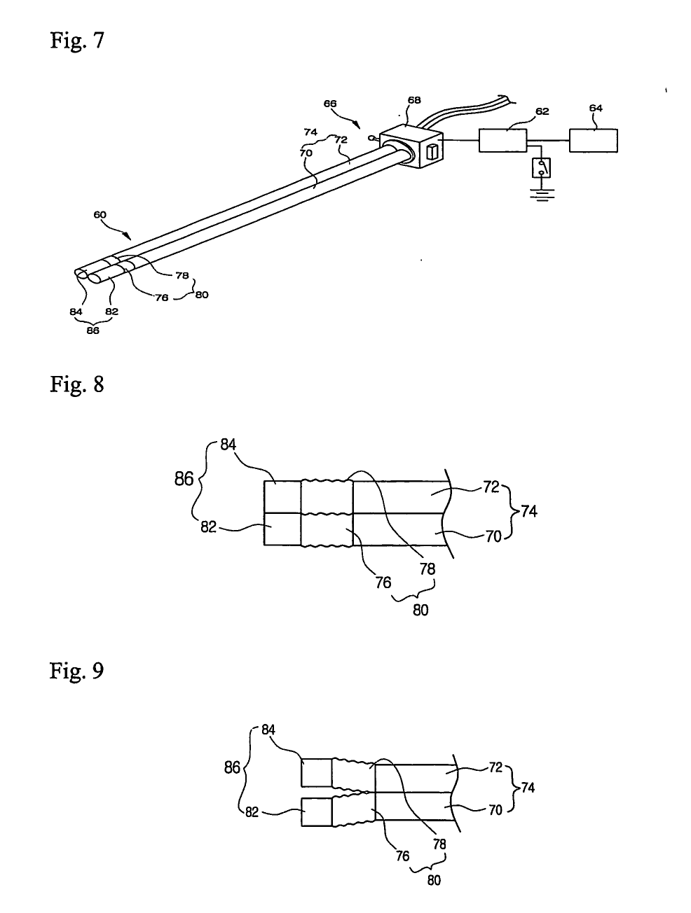

FIG. 6 is a block diagram illustrating a stereoscopic laparoscope apparatus having a spacing adjustable binocular camera assembly in accordance with the present invention. FIG. 7 is a perspective view schematically illustrating the stereoscopic laparoscope apparatus having a spacing adjustable binocular camera assembly in accordance with the present invention. FIGS. 8 and 9 are partially broken away sectional views illustrating states before and after dual left and right cameras are spaced apart from each other, respectively.

As shown in FIGS. 6 to 9, the stereoscopic laparoscope apparatus of the present invention basically comprises a laparoscope 60, a computer 62 for converting and storing image information of the affected part obtained from the laparoscope 60, and a monitor 64 for displaying the image information converted by the computer 62. The stereoscopic laparoscope 60 comprises a supporting unit 74, a flexible tube unit 80, and a spacing adjustable binocular camera assembly 86.

The supporting unit 74 includes a manipulator (L) provided in a body 68 of the laparoscope 60 electrically connected to the computer 62, and a pair of parallel left and right supporting rods 70 and 72 located at one side of the manipulator (L) and having a predetermined length and diameter.

The flexible tube unit 80 includes a pair of left and right flexible tubes 76 and 78, which are adapted to be spaced apart from each other within a predetermined angular range when an actuator 79 is operated by electric signals carried from the manipulator (L) at the tip end of the supporting unit 74.

The binocular camera assembly 86 includes a pair of left and right cameras 82 and 84 installed at the tip end of the flexible tube unit 80, which take images of the affected part in the abdominal cavity through operation of the manipulator (L).

In the above described laparoscope 60, the supporting unit 74 is constructed by making use of two parallel pipes having the same distance as each other and internally provided with electric wires, etc., and at one end of the supporting unit 74 are provided the left and right flexible tubes 76 and 78. The flexible tubes 76 and 78 can be spaced apart from each other within a predetermined angular range as the manipulator (L) is operated by the conventional actuator 79.

To the left and right flexible tubes 76 and 78 are installed the left and right cameras 82 and 84 of the binocular camera assembly 86, respectively.

The binocular camera assembly 86 normally utilizes CCDs, etc., and can increase its definition through pixel upgrade.

The spacing angle between the left and right cameras 82 and 84 of the binocular camera assembly 86 is initially set according to design of the apparatus, and thus does not need additional adjustment by an operator.

Now, the operation of the present invention configured as stated above will be explained.

After the binocular camera assembly 86 of the laparoscope 60 is inserted into the abdominal cavity of the patient, the left and right cameras 82 and 84 are adjusted so that they are spaced apart from each other by the predetermined distance under operation of the actuator 79. In this case, the actuator 79 is operated by means of the manipulator (L) provided in the body 68 of the laparoscope 60.

The fact that the cameras 82 and 84 of the binocular camera assembly 86 are spaced apart from each other means that they can provide a stereoscopic vision similar to that of a human. Therefore, the cameras 82 and 84 can take images of the affected part from different angles within the abdominal cavity, thereby achieving clear stereoscopic images required for the diagnostic, surgical and other medical procedures without problems caused by conventional monocular cameras.

The image information, taken by the left and right cameras 82 and 84 of the binocular camera assembly 86 is stored in the computer 62, and is converted into stereoscopic images so as to be displayed via the monitor 64, thereby providing the three-dimensional clear images to the operator. That is, in addition to solve the problem of conventional monocular cameras, since such left and right cameras 82 and 84 of the binocular camera assembly 86 are adjustable in spacing therebetween by means of the flexible tube unit 80 so as to provide a stereoscopic vision similar to that of a human, resulting in acquisition of stereoscopic images and wide visual observation during laparoscopic surgery, as well as improved definition and convenience in video diagnosis and laparoscopic surgery. Further, the fact that the left and right cameras 82 and 84 are respectively supported by the flexible tubes 76 and 78 enables the cameras 82 and 84 to take images from all directions exceeding four directions, resulting in easy and precise laparoscopic surgery.

1. A stereoscopic laparoscope apparatus comprising a laparoscope, a computer adapted to convert and store image information of the patient's affected part inputted via the laparoscope, and a monitor used to output the image information converted by the computer, the laparoscope comprising:
a supporting unit including a manipulator provided in a body of the laparoscope electrically connected to the computer, and a pair of parallel left and right supporting rods located at one side of the manipulator and having a predetermined length and diameter;
a flexible tube unit including a pair of left and right flexible tubes, which are adapted to be spaced apart from each other within a predetermined angular range according to electric signals generated from the manipulator installed at the tip end of the supporting unit; and
a binocular camera assembly including a pair of left and right cameras installed at the tip end of the flexible tube unit so that they take images of the affected part in the abdominal cavity under operation of the manipulator.

2. The apparatus as set forth in claim 1, wherein the left and right cameras of the binocular camera assembly are arranged so that they are spaced apart from each other within the predetermined angular range as the manipulator is operated upon receiving preset information.
